(19) United States
(12) Patent Application Publication
(10) Pub. No.: US 2013/0144129 A1
(43) Pub. Date: Jun. 6, 2013

(54) Systems and Methods for Monitoring and Encouraging Patient Compliance

(71) Applicant: Carespeak Communications, Inc., East Brunswick, NJ (US)

(72) Inventor: Srdjan Loncar, East Brunswick, NJ (US)

(73) Assignee: Carespeak Communications, Inc., East Brunswick, NJ (US)

(21) Appl. No.: 13/690,798

(22) Filed: Nov. 30, 2012

Related U.S. Application Data
(60) Provisional application No. 61/565,644, filed on Dec. 1, 2011.

(51) Int. Cl. A61B 5/00

(52) U.S. Cl. CPC A61B 5/4833
USPC 600/300

(57) ABSTRACT

Systems and methods for providing information and queries regarding a medical condition to a patient may include receiving, by a processor, an enrollment request from the patient and collecting, by the processor, information about the patient. The enrollment request may include a keyword that corresponds to the medical condition. The systems and methods may further include enrolling, by the processor, the patient in a program that corresponds to the keyword and providing, by the processor, program information from the program to the patient. The program information may include information about the medical condition. The systems and methods may further include querying, by the processor, the patient about whether the patient has completed one or more tasks and based upon a response received from the patient, sending, by the processor, additional information to the patient.

### CROSS REFERENCE TO RELATED APPLICATIONS

This application claims the benefit of priority to U.S. Provisional Patent Application 61/565,644, filed Dec. 1, 2011, and entitled “METHOD FOR MONITORING AND ENCOURAGING A PATIENT TO FILL PERSCRIPTIONS,” the entire contents of which are hereby incorporated by reference.

### BACKGROUND

It is estimated that about 25% to 33% of prescriptions written by physicians are never filled by patients. It is further estimated that up to 75% of patients stop taking their medication within the first year of prescription. Furthermore, about half of those that remain fail to refill their prescriptions. These represent a significant contribution to medication therapy non-compliance that can potentially lead to a deteriorating health of patients, increased costs to the healthcare system, increased costs to employers due to employee sick days, missed revenues for pharmaceutical companies, missed revenues for retail pharmacies and other additional expenses. Similarly, other aspects of medical treatment plans may be ignored by patients. For example, patients may frequently skip doses of medication, forget or choose not to record biometric data such as blood pressure, miss doctor office visits or lab tests and other items included in a medical treatment plan.

### SUMMARY

In an embodiment, a method of providing information and queries regarding a medical condition to a patient may include receiving, by a processor, an enrollment request from the patient and collecting, by the processor, information about the patient. The enrollment request may include a keyword that corresponds to the medical condition. The method may further include enrolling, by the processor, the patient in a program that corresponds to the keyword and providing, by the processor, program information from the program to the patient. The program information may include information about the medical condition. The method may further include querying, by the processor, the patient about whether the patient has completed one or more tasks and sending, by the processor, additional information based upon a response received from the patient.

In an embodiment, a system for providing information and queries regarding a medical condition to a patient may include a processor and a non-transitory, processor-readable storage medium in communication with the processor. The non-transitory, processor-readable storage medium may contain one or more programming instructions that, when executed, cause the processor to receive an enrollment request from the patient and collect information about the patient. The enrollment request may include a keyword that corresponds to the medical condition. The one or more programming instructions that, when executed may further cause the processor to enroll the patient in a program that corresponds to the keyword and provide program information from the program to the patient. The program information may include information about the medical condition. The one or more programming instructions that, when executed may further cause the processor to query the patient about whether the patient has completed one or more tasks and send additional information based upon a response received from the patient.

In an embodiment, a system for providing information and queries regarding a medical condition to a patient may include a processor and a non-transitory, processor-readable storage medium in communication with the processor. The non-transitory, processor-readable storage medium may contain one or more programming instructions that, when executed, cause the processor to receive an enrollment request from the patient and collect information about the patient. The enrollment request may include a keyword that corresponds to the medical condition. The one or more programming instructions that, when executed may further cause the processor to enroll the patient in a program that corresponds to the keyword and provide program information from the program to the patient. The program information may include information about the medical condition. The one or more programming instructions that, when executed may further cause the processor to query the patient about whether the patient has completed one or more tasks, send additional information to the patient based upon a response received from the patient and supply correspondence information to one or more of a healthcare provider and a program manager. The correspondence information may include information regarding the response received from the patient.

### BRIEF DESCRIPTION OF THE DRAWINGS

FIG. 1 depicts a flow diagram of a method that may be used to initialize a patient account for providing reminders according to an embodiment.

FIG. 2 depicts an illustrative screenshot showing an application window that may be used for establishing a keyword for a specific treatment program according to an embodiment.

FIG. 3 depicts an illustrative screenshot showing an application window for setting up content and timing information according to an embodiment.

FIG. 4 depicts an illustrative screenshot showing an application window for setting up an additional module according to an embodiment.

FIG. 5 depicts a flow diagram of a method for enrolling and reminding patients according to various embodiments.

FIG. 6 depicts a flow diagram of an alternative method of monitoring and reporting patient health information and response data for a patient that is enrolled in the program according to an embodiment.

FIG. 7 depicts a block diagram of communications between one or more electronic devices and one or more computing devices according to an embodiment.

FIG. 8 depicts a block diagram of illustrative internal hardware that may be used to contain or implement program instructions according to various embodiments.

FIG. 9 depicts a process for enrolling and tracking a patient according to an embodiment.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

The following terms shall have, for the purposes of this application, the respective meanings set forth below.

An “electronic device” refers to a device, such as, for example, a mobile device, a computing device, a server and one or more components thereof. In some embodiments, the electronic device includes a processor and a tangible, non-transitory, computer-readable memory. The memory may contain programming instructions that, when executed by the processor, cause the computing device to perform one or more operations according to the programming instructions. In some embodiments, the electronic device may be connected to a communications network, such as, for example, the Internet, an intranet, a personal area network, a home area network, a storage area network, a campus area network, a backbone network, a metropolitan area network, a wide area network, a virtual private network and/or the like.

A “mobile device” refers to an electronic device that is generally portable in size and nature. Accordingly, a user may transport a mobile device with relative ease. Examples of mobile devices include, but are not limited to, pagers, cellular phones, feature phones, smartphones, personal digital assistants (PDAs), cameras, tablet computers, phone-tablet hybrid devices (e.g., “phablets”), laptop computers, netbooks, ultrabooks, global positioning satellite (GPS) navigation devices, in-dash automotive components, media players, watches, handheld imaging devices, personal medical devices and/or the like.

A “computing device” is an electronic device and/or a mobile device, such as, for example, a computer or components thereof. The computing device may generally contain a memory or other storage device for housing programming instructions, data or information regarding a plurality of applications, data or information regarding a plurality of electronic devices and/or the like. The programming instructions may be in the form of an application environment, as described in greater detail herein, and/or contain one or more modules, such as software modules for carrying out tasks as described in greater detail herein. The data may optionally be contained on a database, which is stored in the memory or other storage device. The data may optionally be secured by any method now known or later developed for securing data. The computing device may further be in operable communication with one or more electronic devices. The communication between the computing device and each of the one or more electronic devices may further be secured by any method now known or later developed for securing transmissions or other forms of communication.

A “server” is a computing device or component thereof that generally provides data storage capabilities for one or more computing devices. The server can be independently operable from other computing devices and may optionally be configured to store data in a database, a memory or other storage device. The server may optionally contain one or more programming instructions, such as programming instructions in the form of the operating environment, as described in greater detail herein, and/or one or more modules, such as software modules for carrying out processes as described in greater detail herein. The server may have one or more security features to ensure the security of data stored within the memory or other storage device. Examples of security features may include, but are not limited to, encryption features, authentication features, password protection features, redundant data features and/or any other security features now known or later developed. The server may optionally be in operable communication with any of the electronic devices and/or computing devices described herein and may further be secured by any method now known or later developed for securing transmissions or other forms of communication.

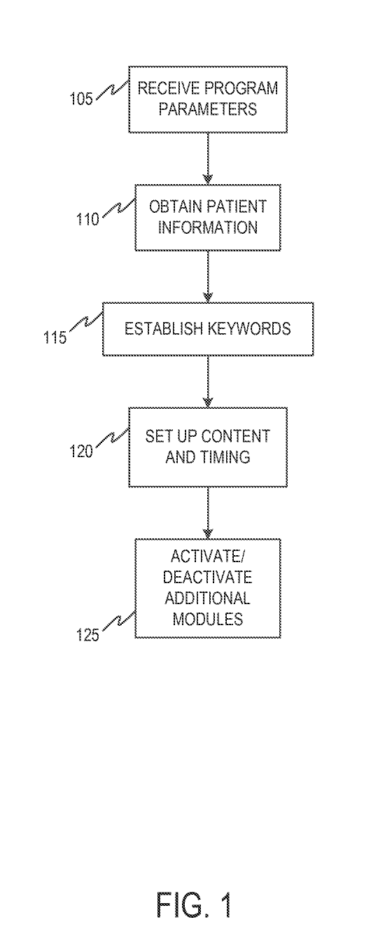

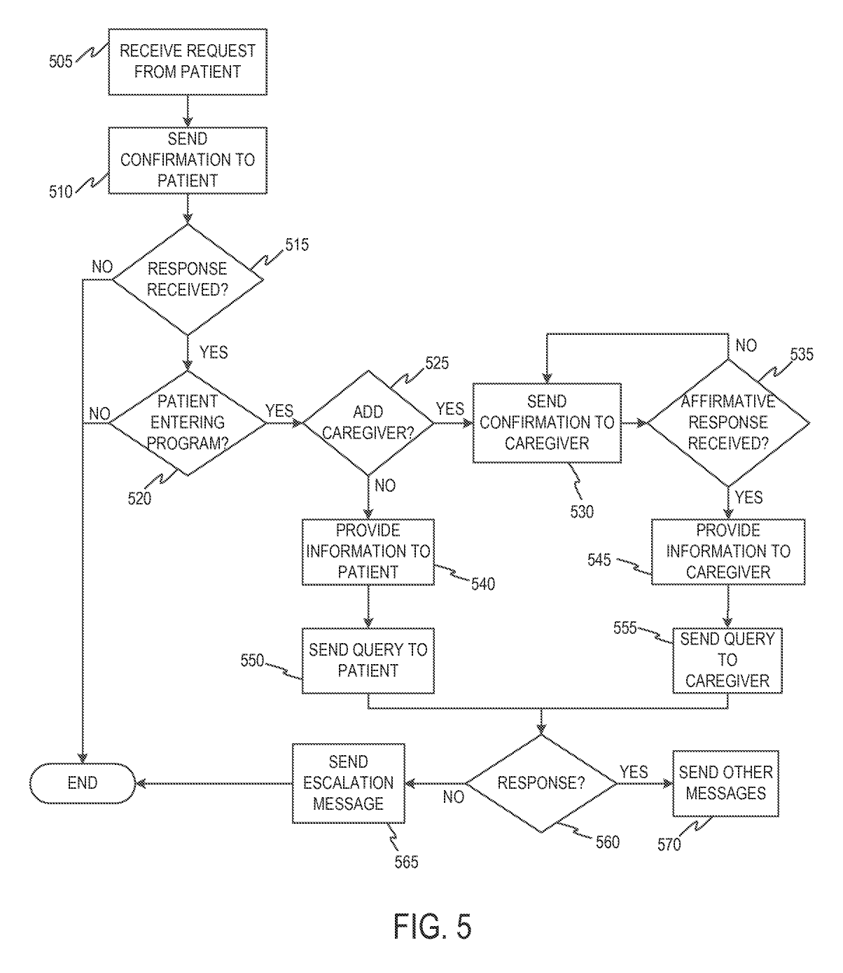

An “application environment” is an embodiment of programming instructions that direct the various components of each electronic device to execute a plurality of operations, such as, for example, those described in more detail in reference to FIGS. 1 and 5. The application environment may generally provide a means for communicating with one or more electronic devices, whether or not explicitly described herein, a means for obtaining data, a means for compiling data, a means for organizing data, a means for transmitting data, a means for performing calculations and a means for completing other tasks, as may be described in greater detail herein.

The present disclosure is directed to a module integrated within a healthcare platform such as the CareSpeak mHealth platform discussed in U.S. Pat. No. 7,956,727, filed Mar. 24, 2008, the content of which is hereby incorporated by reference in its entirety. In one embodiment, the module may be directed to a software application or the like that uses messaging techniques and electronic devices to: (1) allow for patients to be registered for a program; and (2) send factoid messages to registered patients about their conditions and/or medications, send incentives information, provide appointment reminders, provide prescription refill reminders, provide reminders to take medication, provide reminders to record biometric data, provide reminders to respond to surveys and/or the like.

FIG. 1 depicts a flow diagram of a method that may be used to initialize a patient account for providing reminders according to an embodiment of the invention. The embodiment depicted herein is merely illustrative, and those skilled in the art will recognize that other methods may be used without departing from the scope of this disclosure. In various embodiments, the method described herein may be completed with an electronic device, a computing device, a mobile device, a server and/or the like. For purposes of simplicity, the following description will refer to a computing device.

In various embodiments, the computing device may receive 105 program parameters. The program parameters may generally outline the details of a program. The program parameters may generally provide information regarding the type of program to be provided to one or more patients, which includes, but is not limited to, a program for reminding patients to take medication, to refill medication, to attend scheduled medical visits, to enter biometric information, to enter daily living information and/or the like. The program parameters may be received from any entity, including, but not limited to, a medical services provider, a program manager, a clinician, a medical staff person, an administrator, a patient and a caregiver. The program manager may generally be an entity that provides and maintains the program and services discussed herein. The program parameters may be received via an interface, as discussed in greater detail herein. The interface may be accessible via the Internet, a form, a software application and/or the like.

In various embodiments, the computing device may obtain 110 patient information. In some embodiments, the computing device may obtain 110 patient information at the same time it receives 105 the program parameters. In particular embodiments, the computing device may obtain 110 patient information as a subset of receiving 105 the program parameters. In other embodiments, the computing device may obtain 110 patient information separately from receiving 105 the program parameters. The patient information may include any type of patient identifying information, including, but not limited to, name, address, telephone number, email address, mobile number, social security number, sex, age and birthdate. The list may further include other pertinent information for the incentive program, including a patient's health records, a patient's medical history, past and present physicians used by the patient, past and present pharmacists used by the patients, medications currently taken by the patient (both prescription and over-the-counter), medications previously taken by the patient (both prescription and over-the-counter), the patient's biometric data and/or the like. In some embodiments, the computing device may obtain 110 patient information via any text-based communication now known or later developed, including, but not limited to, email, SMS messaging, MMS messaging, GPRS messaging, facsimile messaging, and/or the like. In other embodiments, the computing device may obtain 110 patient information via a form, such as a web-based form and/or a paper-based form. In other embodiments, the computing device may obtain 110 patient information via voice entry, which may use an automated system for detecting and recording voice entries or a person to translate voice entries into text. The information may be collected from the patient, a medical care provider, a family member of the patient, a third party such as an insurance provider and/or the like.

In some embodiments, the patient information may include a list of patients that are potential candidates for the program. In some embodiments, the list of patients may generally include only patients that are determined to be a potential candidate for the program. A patient may be a potential candidate if he/she is referred to the program by a medical services provider, a program manager, a clinician, a medical staff person, an administrator and/or the like. In certain embodiments, the program parameters previously described herein may be individualized for each patient to be enrolled. In other embodiments, the program parameters may be generalized for a group of patients to be enrolled.

In various embodiments, the computing device may establish 115 one or more keywords. The one or more keywords may be automatically established by the computing device, or may be provided to the computing device from a program manager, a physician, a medical services provider, an administrator and/or the like. The one or more keywords may correspond to words that are used by a patient to respond to requests in the program or to sign up for the program, as described in greater detail herein. In some embodiments, the keywords may be received from the patient via a messaging protocol. An example of a messaging protocol may include, but is not limited to, a short message service (SMS) text message, a multimedia service (MMS) text message, an extended messaging service (XMS) text message, an interactive voice response message (IVR), a voicemail, an email, a general packet radio service (GPRS) message and/or the like. In some embodiments, the message may generally be received from the patient's electronic device. In an illustrative example, the computing device may establish “Diovan” as a keyword to be used for hypertension treatment.

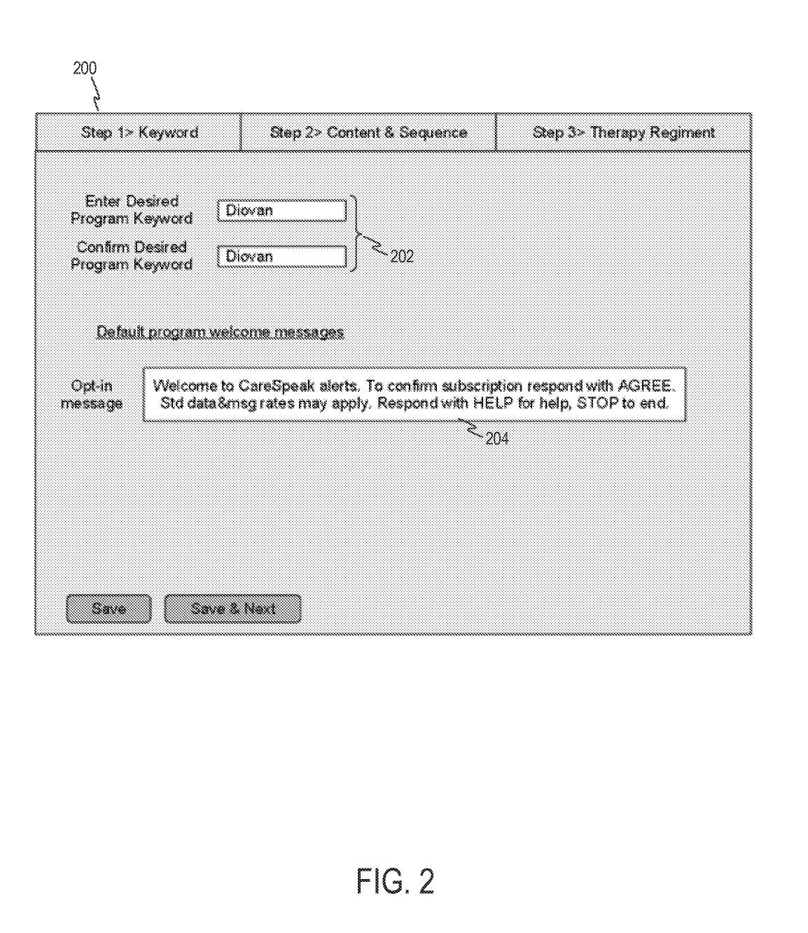

FIG. 2 depicts an illustrative screenshot showing an application window 200 that the computing device may use for establishing 115 (FIG. 1) the keyword for a specific treatment program according to an embodiment. The application window 200 may include a keyword entry area 202 for receiving the keyword. As shown in FIG. 2, the keyword entry area 202 is a text box where the computing device can receive customized text. However, this is shown by way of example only. Other means of receiving data may be used.

The application window 200 may further include a text box 204 where a program manager (PM) can create a message to be sent to each patient that desires to join the program. The message may be selected from a list of existing messages, default to a single message for all programs, or be entered as a custom message by the PM.

Referring back to FIG. 1, in various embodiments, the computing device may set up 120 content and timing information. In some embodiments, the computing device may set up 120 content and timing information by retrieving factoid messages containing an educational or relevant fact about a particular condition or disease. In other embodiments, the computing device may set up 120 content and timing information by receiving factoid messages from an administrator, a PM and/or the like. The factoid may include program information such as, for example, educational messages, motivational messages, facts about the medical condition, facts about a medication, recommendations regarding the medical condition, recommendations regarding a medication and/or the like. The computing device may also receive a message from the PM related to the patient's prescription to tag at the end of the factoid. The computing device may also receive select timing information from the PM that provides instructions for how often and at what time the messages are to be delivered.

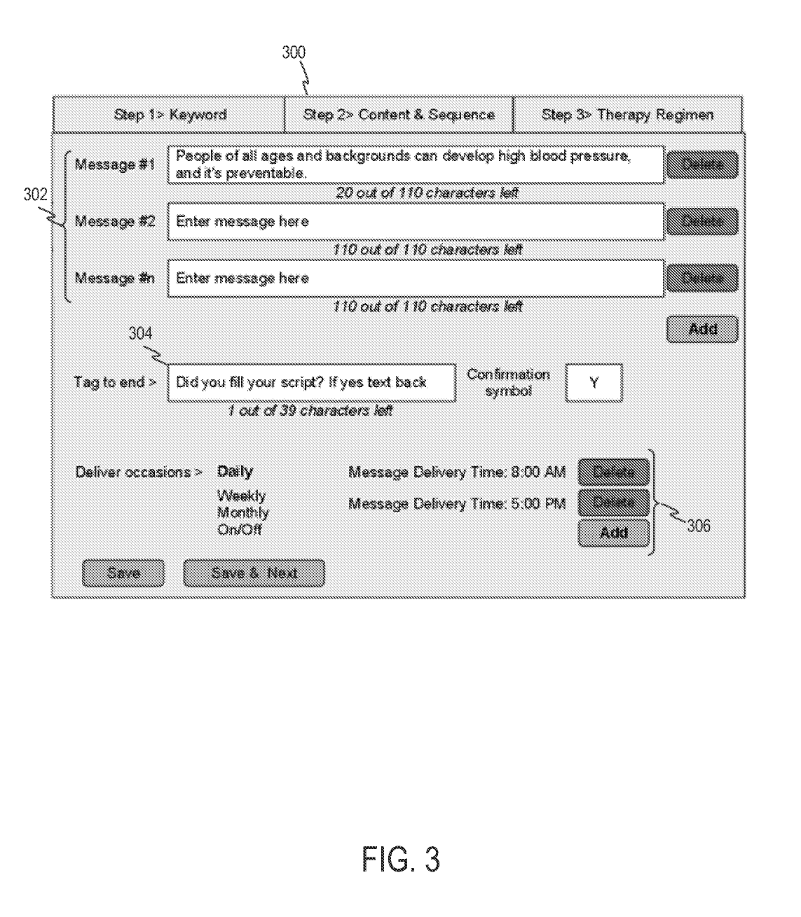

FIG. 3 depicts an illustrative screenshot showing an application window 300 for setting up 120 (FIG. 1) the content and timing information according to an embodiment. The application window 300 may include a message entry area 302 for receiving factoids related to the program. As shown in FIG. 3, the keyword entry area 302 is a text box where the computing device can receive customized text. However, this is shown by way of example only. Other means of receiving data may be used such as a selectable list of messages or a drop-down menu.

The application window 300 may further include a text box 304 where the PM can create a message to be tagged onto the factoids, prompting the user to confirm they have filled their prescription. The message may be selected from a list of existing messages, default to a single message for all programs, or be entered as a custom message by the PM. Additionally, the application window may further include a timing information area 306 where the PM can select the frequency (e.g., daily, weekly, monthly) of message delivery, as well as the specific times when the message is sent.

Referring back to FIG. 1, in various embodiments, the computing device may activate and/or deactivate 125 additional modules. Examples of other modules may include, but are not limited to, medication therapy modules, biometric measurement modules, motivational messaging modules, educational messaging modules, appointment reminder modules, and other similar modules for use with a healthcare platform system. The medication therapy module may include a default regimen for the program (e.g., “1 pill every day at 8:00 AM”) and a refill alert (e.g., every 30 days).

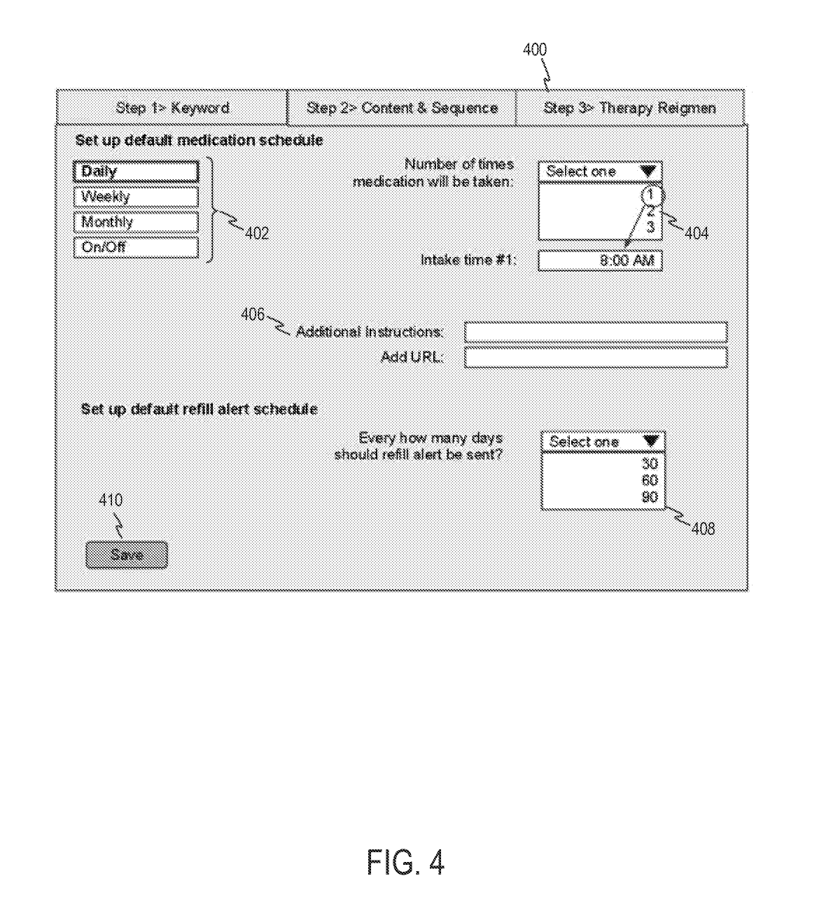

FIG. 4 depicts an illustrative screenshot showing an application window 400 for setting up 112 (FIG. 1) an additional module such as the medication therapy regimen information according to an embodiment. The application window 400 may include a schedule area 402 for selecting a medication schedule. The application window may further include a frequency area 404 where the computing device may receive a number of times the medication is to be taken. The computing device may receive additional instructions or information in a text area 406 as needed. The computing device may also receive a selected value for refill timing in a refill alert scheduling area 408. A save box 410 may be used to complete the program.

FIG. 5 depicts a flow diagram of a method for enrolling and reminding patients according to various embodiments. The embodiment depicted herein is merely illustrative, and those skilled in the art will recognize that other methods may be used without departing from the scope of this disclosure. In various embodiments, the method described herein may be completed with an electronic device, a computing device, a mobile device, a server and/or the like. For purposes of simplicity, the following description will refer to a computing device.

In various embodiments, the computing device may receive 505 a request from the patient. In some embodiments, the request may generally be a request to enroll in a program. The request may be sent in response to a patient being prompted by a doctor, an office staff member, a caregiver or other related person to enter the program. In other embodiments, the request may contain a keyword that identifies the program the patient desires to be enrolled in. The keyword may any keyword previously established, as described in greater detail herein, and may be provided to the patient from a doctor, an office staff member, a caregiver, an insurance company representative, a program manager and/or the like. Based upon the specific program the patient is prompted to join, the keyword received may vary between patients. The computing device may receive 505 the request from the patient via any method of messaging now known or later developed, including, but not limited to, short message service (SMS), multimedia service (MMS), extended messaging service (XMS), an interactive voice response (IVR), a voicemail, an email, a general packet radio service (GPRS) and/or the like.

In various embodiments, the computing device may send 510 a confirmation to the patient to verify that the patient desires to enroll in the program. The confirmation may generally contain a message requesting the recipient to provide a response that confirms enrollment. For example, the confirmation may direct the patient to respond with a “Y” if he/she desires to enroll in the program. The computing device may send 510 the confirmation via any method of messaging now known or later developed, including, but not limited to, short message service (SMS), multimedia service (MMS), extended messaging service (XMS), an interactive voice response (IVR), a voicemail, an email, a general packet radio service (GPRS) and/or the like.

In various embodiments, the computing device may determine 515 whether a response is received. The response may generally be received when the computing device receives any sort of message from the patient. In some embodiments, the response may be affirmative. In other embodiments, the response may be negative. In other embodiments, the response may be neither affirmative nor negative (e.g., an “other” response). If no response is received, the computing device may end the process and fail to send additional messages to the patient.

If the computing device receives 515 a response, it may determine 520 whether the patient is entering the program by determining whether the response is affirmative, negative, or other. If the response is affirmative, the computing device may determine that the patient is enrolling in the program and may proceed to enroll the patient. If the response is negative or other, the computing device may determine that the patient is not enrolling in the program and may end the process.

In various embodiments, once the computing device has enrolled the patient into the program, it may determine 525 whether a caregiver should also be added to the program as well. The caregiver is not limited by this disclosure, and may include, for example, a family member of the patient, a friend of the patient, an in-home healthcare worker and/or the like. In some embodiments, the caregiver may generally obtain information and provide responses to queries on behalf of the patient, as described in greater detail herein. In some embodiments, the computing device may determine 525 that the caregiver is to be added if it is directed to add a caregiver. The computing device may be directed to add a caregiver by the patient, a representative of the patient, a patient's guardian, a medical provider, a program manager, an insurance company representative and/or the like. In other embodiments, the computing device may determine 525 that a caregiver is to be added automatically. In other embodiments, the computing device may determine 525 that a caregiver is to be added based upon an automated review of the patient's medical records.

If the computing device determines 525 that a caregiver is to be added, it may send 530 a confirmation to the caregiver to verify that the caregiver desires to enroll in the program. The confirmation may generally contain a message requesting the recipient to provide a response that confirms enrollment. For example, the confirmation may direct the caregiver to respond with a “Y” if he/she desires to enroll in the program. The computing device may send 525 the confirmation via any method of messaging now known or later developed, including, but not limited to, short message service (SMS), multimedia service (MMS), extended messaging service (XMS), an interactive voice response (IVR), a voicemail, an email, a general packet radio service (GPRS) and/or the like.

In various embodiments, the computing device may determine 535 whether an affirmative response is received. A response may be affirmative if it is not negative or neither affirmative nor negative (“other” response). If no response is received or if an “other” response is received, the computing device may return to send 530 another confirmation to the caregiver after a period of time has elapsed. Periods of time are not limited by this disclosure, and may be, for example, 12 hours, 1 day, 3 days, 5 days, 7 days, 14 days or 30 days. In some embodiments, the computing device may send 530 the confirmation an unlimited number of times until an affirmative response is received from the caregiver. In other embodiments, the computing device may send 530 the confirmation a certain number of times, and if an affirmative response is not received within that number of times, the computing device may end the process with respect to the caregiver and/or complete one or more additional tasks. Examples of the one or more additional tasks include, but are not limited to, sending a message to the patient, sending a message to a program manager, sending a message to an administrator, sending a message to a health care provider and/or the like.

Once the computing device determines 535 that an affirmative response has been received from the caregiver (if applicable) or once it determines 525 that no caregiver is to be added, the computing device may begin providing 540 program information to the patient and/or providing 545 program information to the caregiver. Program information may include, for example, pre-set medication reminders, educational messages, motivation messages, requests for biometric data, facts about the medical condition that the program is focused on, facts about a medication, recommendations regarding the medical condition, recommendations regarding a medication and/or the like to the patient. In some embodiments, the program information may include various factoids as previously discussed herein.

In various embodiments, the computing device may send 550 a query to the patient and/or send 555 a query to the caregiver. The query may generally ask the patient and/or caregiver if he/she completed one or more tasks and request a response from the patient and/or caregiver. Examples of the one or more tasks may include, but are not limited to, taking a medication, filling a prescription, refilling a prescription, attending a medical care appointment, entering biometric information, entering daily living information and/or the like. In an illustrative example, a query may ask: “Did you fill your prescription? If yes, respond with Y.”

In various embodiments, the computing device may determine 560 whether the patient and/or caregiver responds to the query. If the computing device determines 560 that the patient and/or caregiver did not respond, for example, if the patient and/or caregiver sends no response to the query or responds with something other than an affirmative answer within a period of time, the computing device may send 565 an escalation message. The period of time is not limited by this disclosure and may be, for example, 12 hours, 18 hours, 24 hours, 2 days, 3 days, 5 days, 7 days, 10 days or 30 days. The escalation message may be sent to any third party that would want to know if the patient and/or caregiver did not complete the one or more tasks presented in the query. An example of this third party may include, but is not limited to, a healthcare provider, an administrator, an insurance company, a program manager, the caregiver, a family member of the patient a person within a “circle of care” (i.e., health care workers and/or caregivers) and/or the like.

In various embodiments, if the computing device does determine 560 the patient and/or the caregiver has responded to the query, the computing device may optionally send 570 other messages associated with the specific program. The other messages may include, for example, messages such as pre-set medication reminders, additional educational/motivational messages, requests for biometric data, facts about a medical condition, facts about a medication, recommendations regarding a medical condition, recommendations regarding a medication or other similar messages. In some embodiments, the other messages may be sent 570 to the patient and/or caregiver on a regular basis (as determined during set up of the program).

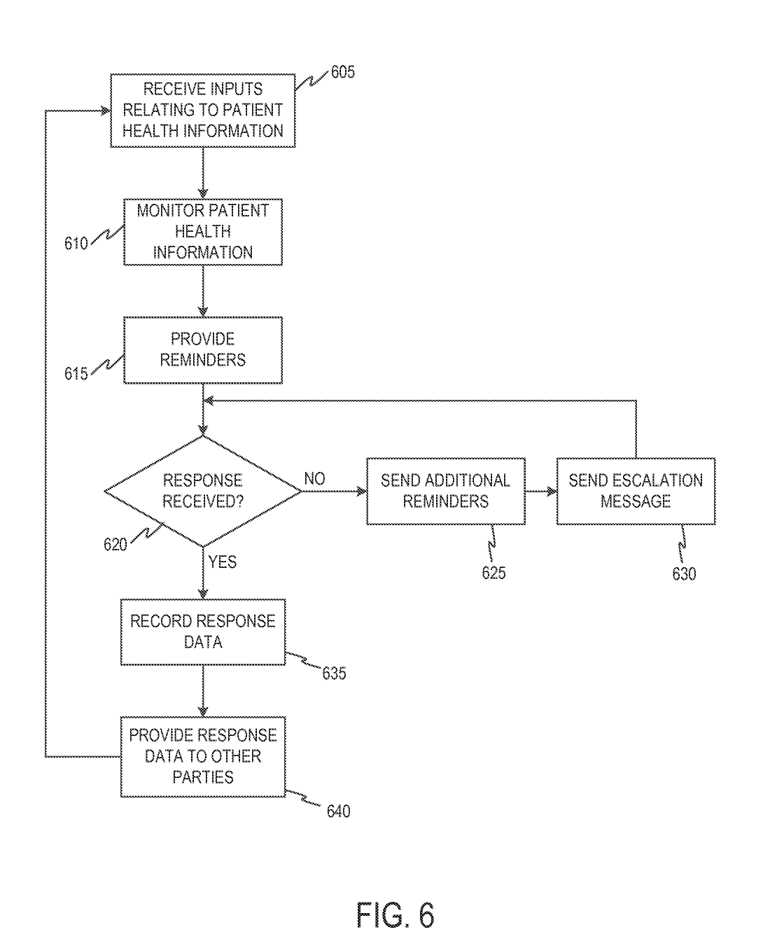

FIG. 6 depicts a flow diagram of an alternative method of monitoring and reporting patient health information and response data for a patient that is enrolled in the program according to an embodiment. The computing device may optionally receive 605 inputs relating to patient health information. The inputs may be received from medical providers, insurance carriers, caregivers, automated data mining systems and/or the like. The patient health information may include, for example, information regarding prescriptions recently obtained, filled and/or refilled by the patient, information regarding recent diagnoses, health update information, and/or the like.

In various embodiments, the computing device may optionally monitor 610 one or more healthcare systems for patient health information, such as hospital databases, medical databases, doctor's office databases, insurance provider databases, and/or other repositories of health information. The monitoring 610 may be completed to supplement or replace inputs received 605 to ensure that the various entities responsible for reporting health information are doing so.

In various embodiments, the computing device may provide 615 one or more reminders to the patient to complete one or more tasks. Examples of tasks that may require reminders include, but are not limited to, taking medication, refilling a prescription, filling a new prescription, attending scheduled healthcare visits, reporting biometric information, reporting everyday living information and/or the like. In addition to providing 615 a reminder, the computing device may request the patient send a response back that verifies that a task has been completed. If no response is received 620, the computing device may send 625 additional reminders to the patient and send 630 an escalation message, as previously described herein.

If a response is received 620 from the patient, the computing device may record 635 the response data. The data may be recorded in a database or the like in addition to the patient health information previously obtained, as described in greater detail herein.

In various embodiments, the computing device may optionally provide 640 response data to other parties. Other parties may include, for example, medical service providers, doctors, nurses, aides, assistants, pharmacists, therapists, hospital personnel, doctor's office personnel, insurance company personnel, caregivers, a family member of the patient and/or the like. In some embodiments, the response data may generally be provided to allow the other parties to keep tabs on a patient to ensure his/her health, to recommend other treatment options and/or other tasks that may be completed for the benefit of the patient.

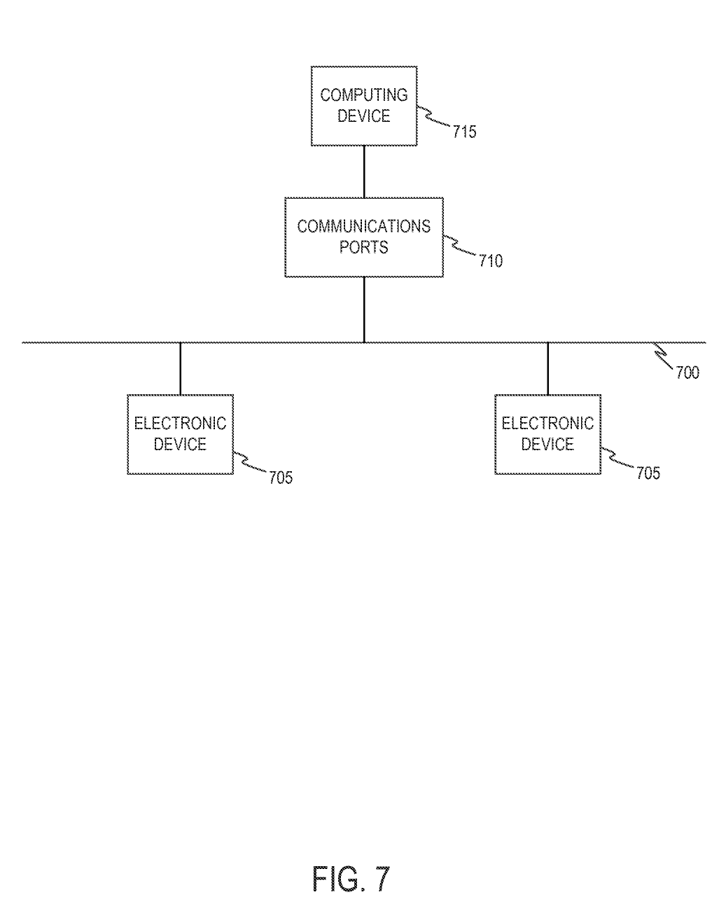

FIG. 7 depicts a block diagram of communications between one or more electronic devices and one or more computing devices according to an embodiment. In this embodiment, the electronic devices may generally be used by one or more patients, whereas the computing device is maintained by another entity such as a program manager (PM) and/or the like. A communications network 700 may serve as an information highway interconnecting the other illustrated components. The communications network is not limited by this disclosure, and may include any communications network now known or later developed. Examples of communications networks may include, but are not limited to, the Internet, intranets, wired networks and wireless networks. One or more electronic devices 705, such as mobile devices, computing devices and the like may connect to the communications network 700. In embodiments where a plurality of electronic devices 705 are connected to the communications network 700, each electronic device 705 may be configured to communicate with other electronic devices via the communications network 700. A computing device 715 may also be connected to the communications network 700, and may optionally connect through the use of one or more communications ports 710.

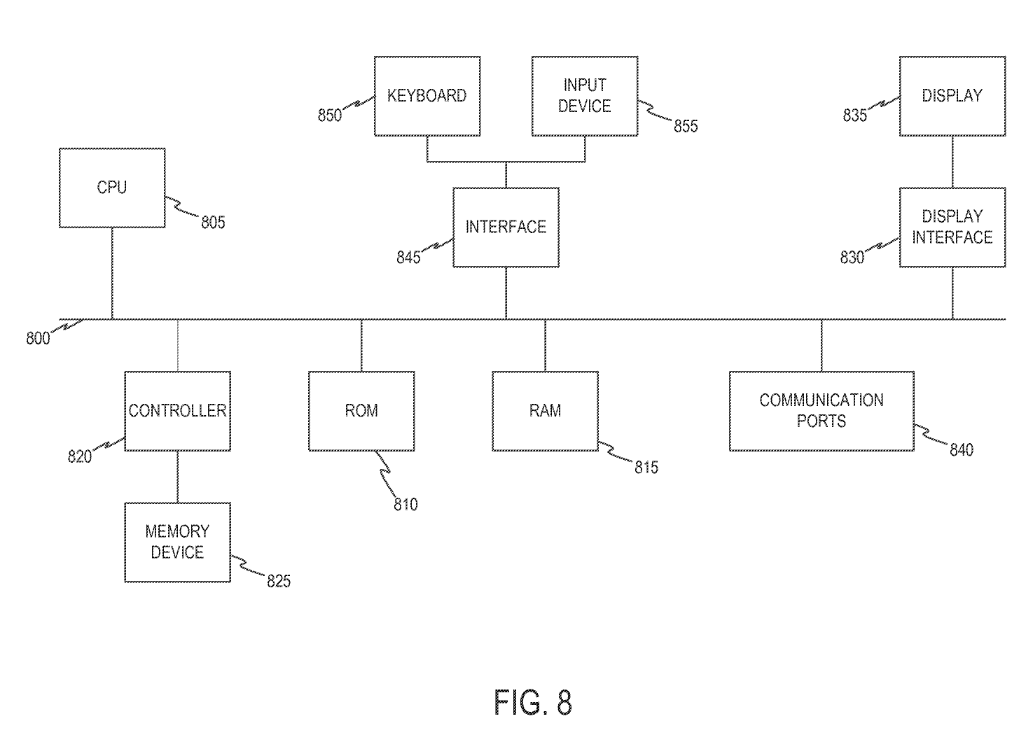

FIG. 8 depicts a block diagram of illustrative internal hardware that may be used to contain or implement program instructions, such as the process steps discussed herein in reference to FIGS. 1-6, according to embodiments. A bus 800 serves as the main information highway interconnecting the other illustrated components of the hardware. CPU 805 is the central processing unit of the system, performing calculations and logic operations required to execute a program. The CPU 805, alone or in conjunction with one or more of the other elements disclosed in FIG. 8, is an illustrative processing device, computing device or processor as such terms are used within this disclosure. Read only memory (ROM) 810 and random access memory (RAM) 815 constitute illustrative memory devices (i.e., processor-readable non-transitory storage media).

A controller 820 interfaces with one or more optional memory devices 825 to the system bus 800. These memory devices 825 may include, for example, an external or internal DVD drive, a CD ROM drive, a hard drive, flash memory, a USB drive or the like. As indicated previously, these various drives and controllers are optional devices.

Program instructions, software, or interactive modules for providing the interface and performing any querying or analysis associated with one or more data sets may be stored in the ROM 810 and/or the RAM 815. Optionally, the program instructions may be stored on a tangible computer-readable medium such as a compact disk, a digital disk, flash memory, a memory card, a USB drive, an optical disc storage medium, such as a Blu-ray™ disc, and/or other non-transitory storage media.

An optional display interface 830 may permit information from the bus 800 to be displayed on the display 835 in audio, visual, graphic, or alphanumeric format. Communication with external devices, such as a print device, may occur using various communication ports 840. An illustrative communication port 840 may be attached to a communications network, such as the Internet, an intranet, or the like.

The hardware may also include an interface 845 which allows for receipt of data from input devices such as a keyboard 850 or other input device 855 such as a mouse, a joystick, a touch screen, a remote control, a pointing device, a video input device and/or an audio input device.

Various embodiments may be realized in the specific example found below.

### Example 1

### Enrolling and Tracking a Patient

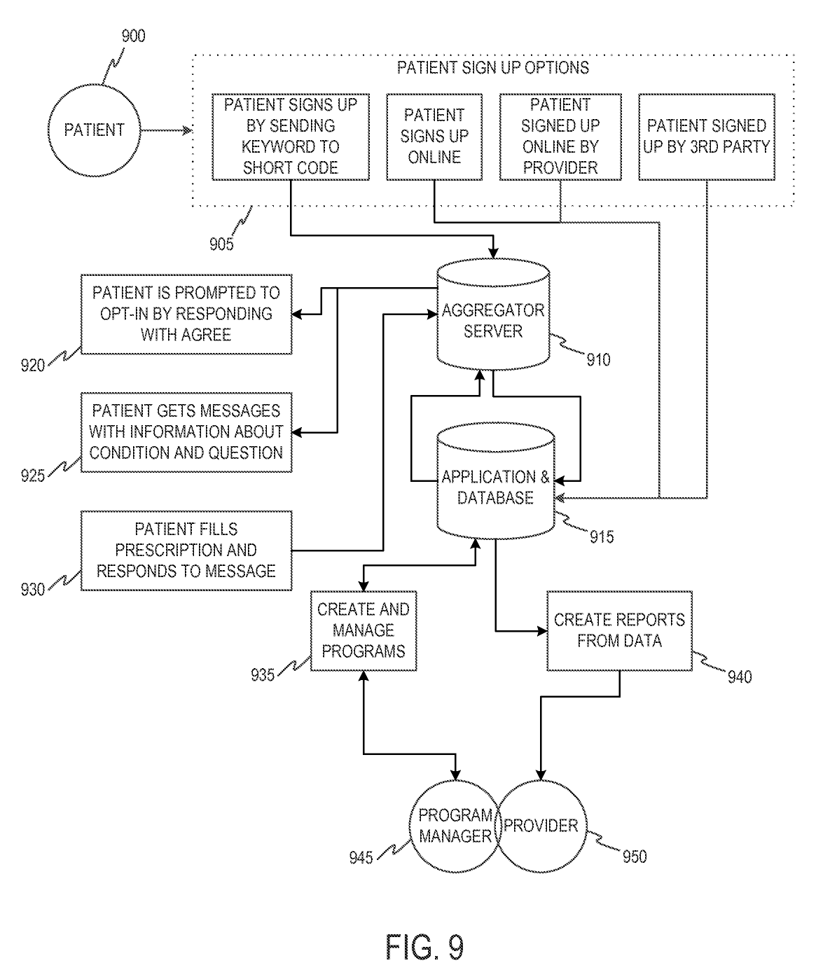

FIG. 9 depicts a process for enrolling and tracking a patient according to an embodiment. The patient 900 may be offered an opportunity to sign up for a text messaging service by his health care provider upon visiting the health care provider's office. The patient 900 is given several sign up options 905: he may choose to use his cell phone to send a keyword to a short code number, he may go online to a web form and sign up, the health care provider may sign him up during the visit, or a third party may automatically sign him up. Once the patient is signed up, the sign up information is sent to an aggregator server 910, which stores the information in a database 915. Alternatively, depending on the method of sign-up, the sign up information may be sent directly to the database 915. The sign up information includes information about the patient, his cell phone number, the diagnosis he received from the health care provider during the visit, and the prescriptions he was given during the visit. The aggregator server 910 may then send a prompt 920 to the patient 900 asking him to agree to opt-in to the service. The prompt 920 is sent via text message from a short code, and the patient 900 is asked to respond with a “Y” to opt in.

Once the patient 900 responds, the aggregator server 910 retrieves information from the application and database 915 and sends text messages 925 to the patient that contain information about his condition, as well as a question that says “Did you fill your script?” Once the patient 900 fills his prescription at the pharmacy, he responds “Y” via text message 930 to the aggregator server 910. The aggregator server 910 may then send the confirmation to the database 915 for storage.

The application and database 915 may create and manage programs 935 as well as create reports 940 from program data and patient data. The created programs 935 may be sent to a program manager 945 for review, implementation and modification. The reports 940 may be sent to the health care provider 950 who can use the reports for future patient visits and diagnoses. Both the program manager 945 and the health care provider 950 may collaborate with the information they received respectively to create stronger and more effective programs and more accurate diagnoses and treatments.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A method for providing information and queries regarding a medical condition to a patient, the method comprising the steps of:
receiving, by a processor, an enrollment request from the patient, wherein the enrollment request comprises a keyword that corresponds to the medical condition;
collecting, by the processor, information about the patient;
enrolling, by the processor, the patient in a program that corresponds to the keyword;
providing, by the processor, program information from the program to the patient, wherein the program information comprises information about the medical condition;
querying, by the processor, the patient about whether the patient has completed one or more tasks; and
based upon a response received from the patient, sending, by the processor, additional information.

2. The method of claim 1, further comprising the steps of:
enrolling, by the processor, a caregiver in the program that corresponds to the keyword;
providing, by the processor, the program information from the program to the caregiver;
querying, by the processor, the caregiver about whether at least one of the patient and the caregiver has completed the one or more tasks; and
based upon a response received from the caregiver, sending, by the processor, additional information

3. The method of claim 1, further comprising the step of:
supplying, by the processor, correspondence information to a healthcare provider, wherein the correspondence information comprises information regarding the response received from the patient.

4. The method of claim 1, further comprising the step of:
supplying, by the processor, correspondence information to a program manager, wherein the correspondence information comprises information regarding the response received from the patient.

5. The method of claim 1, wherein the one or more tasks are selected from a group consisting of taking a medication, filling a prescription, refilling a prescription, attending a medical care appointment, entering biometric information, and entering daily living information.

6. The method of claim 1, wherein the program information further comprises one or more pre-set medication reminders, educational messages, motivational messages, requests for biometric data, facts about the medical condition, facts about a medication, recommendations regarding the medical condition, or recommendations regarding a medication.

7. The method of claim 1, wherein the information about the patient is the patient's name, address, telephone numbers, email addresses, social security numbers, sex, age, birthdate, health records, medical history, past and present physicians, past and present pharmacists, medications currently taken, medications previously taken, biometric data, daily living data, or combinations thereof.

8. The method of claim 1, wherein the enrollment request further comprises a request sent by a web-based form, a paper-based form, a text message, an email, a telephone call, a voicemail, a letter, or a facsimile transmission.

9. The method of claim 1, wherein the program may be customized by at least one of a medical services provider, a program manager, and an administrator, and wherein the program is focused on a single medical condition or illness.

10. A system for providing information and queries regarding a medical condition to a patient, the system comprising:
a processor; and
a non-transitory, processor-readable storage medium in communication with the processor, wherein the non-transitory, processor-readable storage medium contains one or more programming instructions that, when executed, cause the processor to:
receive an enrollment request from the patient, wherein the enrollment request comprises a keyword that corresponds to the medical condition;
collect information about the patient;
enroll the patient in a program that corresponds to the keyword;
provide program information from the program to the patient, wherein the program information comprises information about the medical condition;
query the patient about whether the patient has completed one or more tasks; and
based upon a response received from the patient, send additional information.

11. The system of claim 10, further comprising one or more programming instructions that, when executed, cause the processor to:
enroll a caregiver in the program that corresponds to the keyword;
provide the program information from the program to the caregiver;
query the caregiver about whether at least one of the patient and the caregiver has completed the one or more tasks; and
based upon a response received from the caregiver, send additional information.

12. The system of claim 10, further comprising one or more programming instructions that, when executed, cause the processor to:
supply correspondence information to a healthcare provider, wherein the correspondence information comprises information regarding the response received from the patient.

13. The system of claim 10, further comprising one or more programming instructions that, when executed, cause the processor to:
supply correspondence information to a program manager, wherein the correspondence information comprises information regarding the response received from the patient.

14. The system of claim 10, wherein the one or more tasks are selected from a group of taking a medication, filling a prescription, refilling a prescription, attending a medical care appointment, entering biometric information and entering daily living information.

15. The system of claim 10, wherein the program information further comprises one or more pre-set medication reminders, educational messages, motivational messages, requests for biometric data, facts about the medical condition, facts about a medication, recommendations regarding the medical condition and recommendations regarding a medication.

16. The system of claim 10, wherein the information about the patient comprises the patient's name, address, telephone numbers, email addresses, social security number, sex, age, birthdate, health records, medical history, past and present physicians, past and present pharmacists, medications currently taken, medications previously taken, biometric data, daily living data, or combinations thereof.

17. The system of claim 10, wherein the enrollment request further comprises a request sent by a web-based form, a paper-based form, a text message, an email, a telephone call, a voicemail, a letter, or a facsimile transmission.

18. The system of claim 10, wherein the program may be customized by at least one of a medical services provider, a program manager, and an administrator, and wherein the program is focused on a single medical condition or illness.

19. A system for providing information and queries regarding a medical condition to a patient, the system comprising:
a processor; and
a non-transitory, processor-readable storage medium in communication with the processor, wherein the non-transitory, processor-readable storage medium contains one or more programming instructions that, when executed, cause the processor to:
receive an enrollment request from the patient, wherein the enrollment request comprises a keyword that corresponds to the medical condition;
collect information about the patient;
enroll the patient in a program that corresponds to the keyword;
provide program information from the program to the patient, wherein the program information comprises information about the medical condition;
query the patient about whether the patient has completed one or more tasks;
based upon a response received from the patient, send additional information to the patient; and
supply correspondence information to one or more of a healthcare provider and a program manager, wherein the correspondence information comprises information regarding the response received from the patient.

20. The system of claim 19, further comprising one or more programming instructions that, when executed, cause the processor to:
enroll a caregiver in the program that corresponds to the keyword;
provide the program information from the program to the caregiver;
query the caregiver about whether at least one of the patient and the caregiver has completed the one or more tasks; and
based upon a response received from the caregiver, send additional information.
